(19) United States
(12) Patent Application Publication
(10) Pub. No.: US 2003/0166512 A1
(43) Pub. Date: Sep. 4, 2003

(54) Protein carrier system for therapeutic oligonucleotides

(75) Inventor: Dong Xie, Germantown, MD (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(73) Assignee: MedBridge, Inc.

(21) Appl. No.: 10/365,623

(22) Filed: Feb. 13, 2003

Related U.S. Application Data
(60) Provisional application No. 60/356,053, filed on Feb. 13, 2002.

(51) Int. Cl. A61K 48/00

(52) U.S. Cl. 514/7

(57) ABSTRACT

Therapeutic oligonucleotides, including antisense oligonucleotides and siRNA, are modified with reactive chemical groups connected by flexible linker molecules. The modified oligonucleotides are capable of forming covalent bonds with mobile proteins, in particular with human serum albumin. While retaining biological activity, the resulting complex has enhanced cellular entry, significantly enhanced serum half-life, and reduced immune system stimulation when compared to unmodified oligonucleotides. The modified oligonucleotides overcome many problems associated with current antisense drugs. Modified oligonucleotides of the invention are administered as therapeutic agents, and hybridize to complementary sequences within targeted RNA molecules.

### FIELD OF THE INVENTION

The invention pertains to modified therapeutic oligonucleotides, including antisense oligonucleotides (hereinafter “ASOs”), ribozymes and small interfering RNA (hereinafter “siRNA”), that exhibit enhanced cell entry and extended therapeutic half-life upon forming covalent bonds with mobile proteins. More specifically, the invention pertains to therapeutic oligonucleotides modified with chemically reactive groups capable of forming specific covalent linkages with human mobile proteins in vivo or ex vivo. Additionally, the invention provides methods of introducing therapeutic oligonucleotides into cells, and methods of treating disease conditions using the same.

### BACKGROUND OF THE INVENTION

Therapeutic Oligonucleotides

Therapeutic oligonucleotides, such as ASOs and siRNA, are short segments (e.g., about 7 to about 45 sequence specific bases in length) of single stranded or double stranded DNA or RNA that have been designed to hybridize to a specific sequence on a target DNA or RNA, resulting in the prevention of gene expression, particularly genes shown to contribute to the development of diseases or disorders. There are numerous attributes of ASOs that are attractive from a therapeutic standpoint, including the specificity of ASOs, the ability to prescreen ASOs for non-target hybridization, and the rapidity of development of ASOs as specific therapeutic treatments for known diseases (Tanaka et al., Respir Res., 2:5-9 (2001)). The simplicity and specificity offered by this approach makes the use of therapeutic oligonucleotides such as ASOs an attractive alternative to traditional small molecule or protein-based therapies for the treatment or prevention of diseases or disorders. In particular, the antisense approach may be the fastest way to develop therapeutics utilizing the vast amount of available genomic and cDNA sequence data from human and pathogenic sources.

Nevertheless, several fundamental barriers have been encountered during development of ASOs as broadly applicable therapeutics. One well-known problem is that unmodified ASOs are rapidly degraded by cellular and extracellular nucleases. (Zelphati, O. et al., Res. Dev., 3:323-338 (1993); Thierry, A. R. et al., in Gene Regulation: Biology of Antisense RNA and DNA, Raven Press, N.Y., pp 147-161, (1992)). Modification of the phosphodiester backbone, for example by introduction of phosphorothioate linkages on the DNA backbone, reduces the susceptibility of ASOs to degradation and extends blood half-life from minutes to hours. However, this half-life is still not satisfactory from a therapeutic point of view.

A second problem is the inefficient intracellular delivery of ASOs to target mRNA. Factors contributing to inefficient delivery include: protein binding, mostly to serum albumin; rapid metabolism of the oligonucleotides; and limited ability to cross cell membranes to reach intracellular and/or intranucleus targets. These factors are largely due to the negative charges on ASOs.

A third problem is immune system stimulation by ASOs, which may lead to treatment complications. This immune system stimulation may be associated with systemic toxicity, such as complement-mediated anaphylaxis, altered coagulatory properties, and cytopenia (Galbraith, et al., Antisense Nucl. Acid Drug Des. 4:201-206 (1994)). Often, large and frequent doses are required for efficacy due to the rapid clearance and inefficient delivery of ASOs, and this further facilitates immune system stimulation.

Ribozymes are another form of therapeutic oligonucleotide having potential as a therapeutic. Ribozymes are ribonucleic acids having catalytic activity that can specifically cleave other RNA molecules. Ribozymes as used herein are not limited by size, and can consist of RNA sequences greater than 45 bases in length. Ribozymes function by binding to substrate RNA sequences via Watson-Crick base pairing, followed by cleavage of the phosphodiester backbone of the substrate sequence and inactivation thereof (Bunnell, B. A., et al., Clin. Micro. Rev., 11:42-56 (1998); Jen, K -Y, et al., Stem Cells, 18:307-19 (2000)). One limitation found with ribozymes as a therapeutic is their susceptibility to RNase degradation. Therefore, methods of protecting ribozymes from RNase degradation while retaining the biological activity of ribozymes are useful for the treatment of diseases (Bunnell, B. A., et al., Clin. Micro. Rev., 11:42-56 (1998)).

Carrier Protein Conjugates

The use of mobile proteins, such as blood proteins, as carrier molecules for therapeutics is broadly known in the art. In numerous studies peptides and small molecule drugs are modified with reactive groups to enable conjugation to mobile proteins. Mobile proteins of the invention include, but are not limited to, serum albumin, globulins, transferrin, immunoglobulins, ferritin, and the like. Molecules conjugated to mobile proteins may retain their original therapeutic activity, yet exhibit significantly longer half-life and improved bioavailability. The conjugation can be nonspecific (e.g., using N-hydroxysuccinimide which reacts with available amino groups in the mobile protein) or specific (e.g., using maleimido groups which react with a single free thiol group in albumin (See U.S. Pat. No. 6,329,336 for example)). The conjugation has been completed in vivo or ex vivo, although in vivo conjugation may have certain advantages. Typical applications of these conjugates have been focused on extracellular or cell surface targets. Thus the conjugated therapeutics in such applications do not have to travel through the cell membrane. It is unclear whether this approach can be applied to intracellular targets.

There is a need in the art for therapeutic compounds that readily cross cellular membranes and deliver active therapeutics, such as ASOs, siRNA and ribozymes, to intracellular targets such as RNA or DNA targets. The instant invention fulfills these and more needs in the art by providing novel therapeutic compounds that deliver active therapeutic oligonucleotides intracellularly, while also retaining an extended systemic half-life. The invention further provides methods of treating diseases and/or disorders using therapeutic oligonucleotide compounds of the invention.

### SUMMARY OF THE INVENTION

The invention comprises compounds, means and methods, which together enable the use of novel, chemically reactive derivatives of therapeutic oligonucleotides that can react with available functionalities on mobile proteins, including mobile blood proteins, to form covalent linkages. The invention also provides a method of using therapeutic oligonucleotide derivatives having reactive groups to enhance cellular entry, to resist nuclease degradation and improve in vivo half-life, and to selectively block the expression of a particular gene or genes. In one embodiment, the invention encompasses novel chemically reactive derivatives of ASOs that contain a chemically reactive group conjugated to the ASOs through a linker molecule. These modified ASOs can react with amino groups, hydroxyl groups or thiol groups on mobile proteins, in particular human serum albumin, to form stable covalent bonds.

In another embodiment, the invention encompasses novel chemical reactive derivatives of siRNAs that contain a chemically reactive group conjugated to the siRNAs through a linker molecule. These modified siRNAs can react with amino acids, hydroxyl groups or thiol groups on mobile proteins, in particular human serum albumin, to form stable covalent bonds.

In another embodiment, the invention encompasses novel chemical reactive derivatives of ribozymes that contain a chemically reactive group conjugated to the ribozymes through a linker molecule. These modified ribozymes can react with amino acids, hydroxyl groups or thiol groups on mobile proteins, in particular human serum albumin, to form stable covalent bonds.

The invention provides a method of enhancing cellular entry and improving half-life using compounds comprising modified therapeutic oligonucleotides, by the steps of a) mixing compounds (in vivo or ex vivo) with a biological sample containing either endogenous blood or tissue fluid, or recombinant mobile proteins; b) conjugating to mobile proteins present in the biological sample; c) uptake of the conjugates into cells and allowing binding of the therapeutic oligonucleotide to intracellular and intranucleus targets; and d) selectively blocking the expression of a particular gene.

The invention further provides methods for treating disease conditions using therapeutic oligonucleotides of the invention and their derivatives, by the steps of a) mixing compounds (in vivo or ex vivo) with a biological sample containing either endogenous blood or tissue fluid, or recombinant mobile proteins; b) conjugating to mobile proteins present in the biological sample; c) uptake of the conjugates into cells and allowing binding of the therapeutic oligonucleotide to intracellular and intranucleus targets; and e) selectively blocking the expression of a particular gene.

### BRIEF DESCRIPTION OF THE FIGURES

FIG. 1 shows the general structure of ASOs containing a 5′-(BMPS)(C6NH) linker and maleimide reactive moiety. BMPS means N-(β-maleimidopropyloxy) succinimide ester.

FIG. 2 shows the structure of the N3_{—}9S-MPA linker, which may be used to conjugate to an siRNA duplex.

### DETAILED DESCRIPTION OF THE EMBODIMENT

Definitions

As used herein, “oligonucleotides” shall mean single or double stranded RNA or DNA, including ASOs and siRNA capable of binding to complementary single or double stranded RNA or DNA target sequences. The sequence-specific portion of the therapeutic oligonucleotides that are ASOs or siRNA of the invention comprise nucleotide sequences of from about 7 bases to about 45 bases in length. Additional bases that are not sequence-specific may be included in the oligonucleotides, such as for example linker sequence. By sequence-specific is meant the portion of the oligonucleotide that is complementary to the target RNA or DNA and/or directs cleavage of the target RNA or DNA.

As used herein, “ASOs” shall mean short stretches (about 7 to about 45 sequence-specific nucleotides) of DNA or derivatized DNA (e.g., phosphorothioated DNA) that contains sequence which is complementary to a target DNA or RNA. The complementary portion of the ASOs will typically range from about 30% to about 100% of the oligonucleotide.

As used herein, “siRNA” shall mean an RNA duplex in which each strand of the duplex contains between about 15 and about 30 bases in length, and wherein at least one of the strands shares at least about 90%, more preferably up to about 100% homology with a DNA or RNA target.

As used herein, “ribozyme” shall mean ribonucleic acid having catalytic activity that can specifically cleave other RNA molecules. As used in the present invention, therapeutic oligonucleotides that are ribozymes are not limited by size.

As used herein, “mobile proteins” shall mean proteins that do not have a fixed site for any extended period of time, generally not exceeding five minutes, or more usually one minute. “Fixed sites” do not include intracellular localization. Mobile proteins are not membrane-associated and are systemically distributed for extended periods of time. Examples of mobile proteins include, but are not limited to, circulating albumins such as human serum albumin, human transferrin, globulins, immunoglobulins such as IgG, and variants thereof having virtually the same physical or chemical characteristics. It is understood that mobile proteins may have different functions, such as immunoglobulins which bind different targets. It is also understood that variant and/or mutant forms of these mobile proteins may be present in the body. These functional and/or chemical variants and/or mutants are also encompassed by mobile proteins of the invention.

As used herein, “gene expression” shall mean mRNA synthesis or mRNA translation.

In the broadest sense, the objectives of the invention are accomplished by modifying therapeutic oligonucleotides with chemical reactive groups so that the therapeutic oligonucleotides conjugate to naturally occurring mobile proteins which enhance cellular entry and resist extracellular nuclease cleavage. In one embodiment of the invention, the chemical reactive groups are conjugated to therapeutic oligonucleotides through linker molecules that provide degrees of flexibility. The site of modification of the therapeutic oligonucleotide with a chemical reactive group is selected so that it will not affect the biological activity.

In a preferred embodiment of the invention, the therapeutic oligonucleotide is modified with a chemical reactive group at the 5′ or 3′ terminus of the oligonucleotide. Injection of terminally modified therapeutic oligonucleotides into blood or tissue results in rapid covalent linkage of these agents with mobile proteins.

The prior art has shown that the modification of some agents, for example therapeutic peptides, with chemical reactive groups enables the formation of covalent bonds with mobile proteins, thereby increasing the half-life of the resulting protein conjugates (See U.S. Pat. No. 5,612,034; U.S. Pat. 6,103,233; U.S. Pat. No. 6,107,489; and U.S. Pat. No. 6,329,336). The proposed prior art targets for the protein conjugates, as well as conjugates of other active agents of interest were extracellular and blood-related targets. However, it was unknown whether the attributes found with these conjugates would necessarily extend to the therapeutic oligonucleotides of the present invention. For example, therapeutic proteins and therapeutic oligonucleotides encounter numerous distinct problems upon administration into the body. By way of example, therapeutic proteins may elicit a greater immune response than therapeutic oligonucleotides. In contrast, therapeutic oligonucleotides often suffer nuclease degradation upon administration as noted supra. Similarly, therapeutic oligonucleotides may bind non-specifically to endogenous compounds such as proteins or other nucleic acids.

The present inventor has discovered that therapeutic oligonucleotides of the invention benefit from improved half-lives in vivo upon conjugation to mobile proteins. Additionally, it has been discovered that therapeutic oligonucleotides of the invention retain therapeutic efficacy following conjugation with mobile proteins. Most importantly, it has been discovered that cellular entry of the therapeutic oligonucleotides of the invention is enhanced through conjugation to mobile proteins.

As a particularly important distinction from the prior art, conjugation of the terminally modified therapeutic oligonucleotides to mobile proteins substantially enhances cellular entry of the therapeutic oligonucleotides, while retaining the binding affinity to the target DNA or RNA. This is particularly important, as the purpose of the invention is to introduce therapeutic oligonucleotides to intracellular and/or intranuclear targets.

Conjugation of the terminally modified therapeutic oligonucleotides to mobile proteins also renders the oligonucleotides markedly resistant to intracellular and extracellular degradation by nucleases. It is believed that this property is primarily attributable to the conjugation of the therapeutic oligonucleotide to the mobile blood proteins, such as serum albumin.

Covalent linkage of therapeutic oligonucleotides to mobile proteins, particularly human serum albumin, reduces undesired immune responses to these therapeutic agents in many cases. Therapeutic oligonucleotides of the invention retain efficacy upon covalent linkage to mobile proteins, allowing for example hybridization of therapeutic DNA oligonucleotides with complementary RNA sequences, forming substrates that are recognized and cleaved by RNaseH. As a result of the cleavage of the RNaseH substrate, expression of the corresponding gene is selectively blocked. Such selective inhibition of gene expression is desired in various therapeutic applications.

Nucleic Acids

Therapeutic oligonucleotides of the invention are single or double stranded RNA or DNA oligonucleotides capable of binding target RNA or DNA sequences, including endogenous regulatory sequences, thereby inhibiting gene expression.

In one embodiment of the invention, therapeutic oligonucleotides of the invention are ASOs. ASOs encompass single-stranded DNA or RNA that is complementary to a portion of a specific RNA sequence, or alternatively the complementary gene sequence, and reduce or inhibit gene expression. Non-limiting examples of ASOs include RNA sequences complementary to an mRNA transcript, thereby forming an RNA duplex resulting in reduced levels of translation. Alternatively, an ASO may encompass a DNA sequence complementary to an mRNA transcript, which hybridizes with the mRNA transcript and serves as a substrate for RNaseH.

The technology of antisense oligonucleotides has been known in the art as a promising source of therapeutics. Antisense oligonucleotides rely upon Watson-Crick base pairing between a known nucleic acid sequence and its reverse complement to inhibit gene expression (Jen, K., et al., Stem Cells, 18:307-19 (2000)). Antisense oligonucleotide therapy can be utilized to combat a wide range of disorders, for example the expression of human genes involved in diseases or disorders, or alternatively by targeting the replication of infectious agents (Tanaka, M., et al., Respir. Res., 2:5-9 (2000); Bunnell, B. A., et al., Clin. Micro. Rev., 11:42-56 (1998)). Crucial considerations which must be addressed when designing antisense oligonucleotide therapies include antisense stability in vivo, effective delivery of the antisense oligonucleotide therapeutic, and efficient intracellular localization of the antisense oligonucleotide (Jen, K., et al., Stem Cells, 18:307-19 (2000)).

It is well known that, depending on the target gene, ASOs which hybridize to any part of the target gene, such as coding regions, introns, the 5′ untranslated region (5′UTR), translation initiation site, or 3′UTR may have therapeutic utility. Therefore, the sequences listed herein are merely exemplary of the possible therapeutic oligonucleotides that may be used with the invention, which include all of the ASOs known in the art. Furthermore, all of the alternative nucleic acid chemistries proposed in the art can be used with the invention although the degree of effectiveness may vary. Chemistries applicable with the therapeutic oligonucleotides of the invention are discussed in further detail in the section entitled “Conjugation Chemistry and Carrier Molecules” provided infra. In short, the compounds listed herein represent the broad class of therapeutic oligonucleotides of various chemistries which are useful with this invention. In one embodiment of the invention, the sequence-binding portion of ASO and siRNA therapeutic oligonucleotides of the invention is about 7 to about 45 bases in length. In a preferred embodiment of the invention, the sequence-binding portion of ASO and siRNA therapeutic oligonucleotides of the invention is about 10 to about 30 nucleotides in length. In a particularly preferred embodiment of the invention, the sequence-binding portion of ASO and siRNA therapeutic oligonucleotides of the invention is about 15 to about 25 nucleotides in length. Additional oligonucleotides which are useful in the invention include oligonucleotides previously demonstrating efficacy in free form in the art. Ribozyme therapeutic oligonucleotides of the invention are not restricted by size.

Therapeutic oligonucleotides of the invention also encompass siRNA. siRNA derive from RNA interference, which is a natural cellular process for silencing the transcription of certain genes (Sharp, P. A., Genes & Dev., 15:485-490 (2001); Carmichael, G. G., Nature, 418:379-380 (2002)). siRNA associate with cellular protein complexes and direct cleavage of complementary target RNAs by those protein complexes.

In the present invention, siRNA encompass duplex RNAs of approximately 15-30 bases in length, one strand of the duplex RNA preferably having at least about 90% homology with a RNA target, more preferably having up to about 100% homology with a RNA target. Alternatively, siRNAs share enough homology with a RNA target to direct cleavage of complementary target RNA by protein complexes. Homology between two nucleotide sequences can be determined by one of ordinary skill in the art using search-based computer programs, such as the BLAST or FASTA programs. Alternatively, one of ordinary skill in the art can determine sequence homology using sequence alignment programs such as MegAlign (contained within the DNASTAR suite of computer programs).

siRNAs are modified with chemical reactive groups described infra, enabling the formation of covalent bonds with mobile proteins, preferably human serum albumin. In a preferred embodiment of the invention, modification of the siRNA duplex through addition of a chemical reactive group occurs at a terminus. Chemical modification of the RNA duplex with a chemical reactive group may occur at any of the 4 termini of the RNA duplex, either the 5′ or 3′ termini of either of the two RNA strands of the RNA duplex.

Therapeutic oligonucleotides of the invention further encompass derivatized DNAs. Derivatized DNAs include, but are not limited to, DNAs having modified backbones such as phosphorothioated DNAs, which are synthesized by replacing phosphodiester linkages with phosphorothioate linkages. Additional DNA derivatives of the invention encompass methylphosphonate (Miller and Ts'o, Anticancer Drug Des., 2:117-128 (1987)), and peptide linkages (e.g., PNAs)(Bonham et al., Nucleic Acid Res., 23:1197-1203 (1995)) to enhance resistance to nuclease degradation; and modifications to the nucleoside base, such as C5-propynyl-dU,dC modified oligonucleotides (Wagner et al., Science. 260:1510-1512 (1993)) and “G-clamp”-modified deoxycytosines (Flaganan et al., Proc. Natl. Acad. Sci. USA, 96:3513-3518 (1999)), to improve mRNA binding affinity.

In one embodiment of the invention, DNA derivatives are therapeutic oligonucleotides having phosphorothioate linkages that resist nuclease degradation and permit RNaseH cleavage of the target mRNA. Phosphorothioated DNA employ a phosphothioate linkage as opposed to a phosphodiester linkage during synthesis of the therapeutic oligonucleotide. Phosphothioated oligonucleotides of the invention can be synthesized, for example, using an automated DNA synthesizer. The thioating reagent 3H-1,2-benzodithiole-3-one, 1,1-dioxide is commonly used to generate the thiol bond located within the phosphorothioated linkage. The entire therapeutic oligonucleotide, or alternatively, a portion of the therapeutic oligonucleotide may be phosphothioated (See, for example, Gait, M. J., “Oligonucleotide Synthesis, a practical approach”, Oxford Univ. Press, New York (1984); Reddy, M. P. et al., Tetrahedron Lett., 35(25):4311-14 (1994)). In a particular embodiment of the invention, the non-conjugated terminus, or in the case of an oligonucleotide duplex the non-conjugated termini, of the therapeutic oligonucleotide is phosphothioated.

In another embodiment of the invention, DNA derivatives are therapeutic oligonucleotides that are designed to contain locked nucleic acids (hereinafter “LNAs”), as described in the literature (Kurreck, J., et al., Nuc. Acid Res., 30:1911-18 (2002). Therapeutic oligonucleotides containing LNAs may have, among other attributes, improved affinity for complementary sequences and increased melting temperatures (hereinafter “Tm”).

The therapeutic oligonucleotides of the invention may be derived from any number of sources, including genomic DNA, cDNA, mRNA, and synthetic oligonucleotides. Therapeutic oligonucleotides further include oligonucleotides containing nucleic acid analogs, such as for example phosphorothioated antisense oligonucleotide derivatives described by Stein, et al. (Science 261:1004-1011 (1993)) and the derivative phosphorothioated oligonucleotides described in U.S. Pat. Nos. 5,264,423 and 5,276,019, the disclosures of each of which are herein incorporated by reference. Preferably, oligonucleotides containing nucleic acid analogs possess at least some of the following beneficial attributes, namely resistance to cleavage by nucleases, good aqueous stability, and efficient hybridization with complementary DNA sequences. Furthermore, in another embodiment the therapeutic oligonucleotides of the invention comprise or are complementary to transcriptional and translational regulatory sequences, including promoter sequences and enhancer sequences.

In a particularly preferred embodiment of the invention, therapeutic ASO sequences (putatively target specific) that are useful in this invention include, but are not limited to, the following ASO sequences (oriented 5′ to 3′): 1.) murine ICAM-1 (Intracellular Adhesion Molecule-1) (phosphorothioate)

| | | |
|---|---|---|
| 1.) | murine ICAM-1 (Intracellular Adhesion Molecule-1) | |
| | (phosphorothioate) | |
| | TGCATCCCCCAGGCCACCAT | (SEQ ID NO:1) |
| 2.) | murine ICAM-1 | |
| | (phosphodiester) | |
| | TGCATCCCCCAGGCCACCAT | (SEQ ID NO:1) |
| 3.) | human ICAM-1 | |
| | (phosphorothiote) | |
| | GCCCAAGCTGGCATCCGTCA | (SEQ ID NO:2) |
| 4.) | human erb-B-2 gene | |
| | (phosphodiester or phosphorothioate) | |
| | GGTGCTCACTGCGGC | (SEQ ID NO:3) |
| 5.) | human c-myc gene | |
| | (phosphorothioate) | |
| | AACCGTTGAGGGGCAT | (SEQ ID NO:4) |
| 6.) | human c-myc gene | |
| | (phosphodiester) | |
| | AACGTTGAGGGGCAT | (SEQ ID NO:5) |
| 7.) | human c-myc gene | |
| | (phosphodiester or phosphorothioate) | |
| | TAACGTTGAGGGGCAT | (SEQ ID NO:6) |
| 8.) | human c-myb gene | |
| | (phosphodiester or phosphorothioate) | |
| | TATGCTGTGCCGGGGTCTTCGGGC | (SEQ ID NO:7) |
| 9.) | human c-myb gene | |
| | (phosphodiester or phosphorothioate) | |
| | GTGCCGGGGTCTTCGGGC | (SEQ ID NO:8) |
| 10.) | human IGF-1R (Insulin Growth Factor 1 - Receptor) | |
| | (phosphodiester or phosphorothioate) | |
| | GGACCCTCCTCCGGAGCC | (SEQ ID NO:9) |
| 11.) | human IGF-1R | |
| | (phosphodiester or phosphorothioate) | |
| | TCCTCCGGAGCCAGACTT | (SEQ ID NO:10) |
| 12.) | human EGFR (Epidermal Growth Factor Receptor) | |
| | (phosphodiester or phosphorothioate) | |
| | CCGTGGTCATGCTCC | (SEQ ID NO:11) |
| 13.) | human VEGF (Vascular Endothelial Growth Factor) gene | |
| | (phosphodiester or phosphorothioate) | |
| | CAGCCTGGCTCACCGCCTTGG | (SEQ ID NO:12) |
| 14.) | murine PKC-alpha (Phosphokinase C - alpha) gene | |
| | (phosphodiester or phosphorothioate) | |
| | CAGCCATGGTTCCCCCCAAC | (SEQ ID NO:13) |
| 15.) | human PKC-alpha | |
| | (phosphodiester or phosphorothioate) | |
| | GTTCTCGCTGGTGAGTTTCA | (SEQ ID NO:14) |
| 16.) | human bcl-2 gene | |
| | (phosphodiester or phosphorothioate) | |
| | TCTCCCAGCGTGCGCCAT | (SEQ ID NO:15) |
| 17.) | human c-raf-1 protein kinase | |
| | (phosphodiester or phosphorothioate) | |
| | GTGCTCCATTGATGC | (SEQ ID NO:16) |
| 18.) | human VEGFR-1 (Vascular Endothelial Growth Factor Receptor 1) | |
| | ribozyme | |
| | GAGUUCUGAUGAGGCCGAAAGGCCGAAAGUCUG | (SEQ ID NO:17) |
| 19.) | P53 Oncogene | |
| | CCCTGCTCCCCCCTGGCTCC | (SEQ ID NO:18) |
| 20.) | Human urokinase receptor | |
| | CGGCGGGTGACCCATGTC | (SEQ ID NO:19) |
| 21.) | HIV-1 (human immunodeficiency virus type 1) | |
| | TCTTCCTCTCTCTACCCACGCTCTC | (SEQ ID NO:20) |
| 22.) | MB-003 | |
| | AAAGTATCCCAGCCGCCGTT | (SEQ ID NO:21) |
| 23.) | MB-006 | |
| | TCCCGGTTGCTCTGAGACAT | (SEQ ID NO:22) |

Additionally, the modified therapeutic oligonucleotides of the invention may include one or more labels (e.g., radioactive label, biotin, fluorescent label, chemiluminescent or colorimetric label) for the purpose of providing clinical diagnosis relating to the presence or absence of complementary nucleic acids or for monitoring therapy with the therapeutic oligonucleotides.

The modified therapeutic oligonucleotides are generally administered parenterally, such as intravascularly (IV), intraarterially (IA), intramuscularly (IM), subcutaneously (SC), or the like. Administration may also be made by transfusion. In some instances, where reaction of the functional group is relatively slow, administration may be oral, nasal, rectal, transdermal or aerosol, where the nature of the conjugate allows for transfer to the vascular system. Usually a single injection will be employed, although more than one injection may be used, if desired. The modified therapeutic oligonucleotides may be administered by any convenient means, including syringe, trocar, catheter, or the like. The particular manner of administration will vary depending upon the concentration to be administered, whether a single bolus or continuous administration, or the like. Preferably, the administration will be intravascularly, where the site of introduction is not critical to this invention, preferably at a site where there is rapid blood flow, (e.g., intravenously, peripheral or central vein). Other routes may find use where the administration is coupled with slow release techniques or a protective matrix. The intent is that the therapeutic oligonucleotides are effectively distributed in the blood, so as to be able to react with the mobile proteins. The concentration of the modified oligonucleotide for administration will vary, generally ranging from about 1 pg/ml to 100 mg/ml, pre-administration. The total amount administered intravascularly will generally be in the range of about 0.1 mg to about 500 mg, more usually about 1 mg to about 250 mg.

Conjugation Chemistry and Carrier Molecules

The therapeutic oligonucleotides of the invention form covalent bonds in vivo or ex vivo to various mobile proteins through reactive groups conjugated to the therapeutic oligonucleotides. The reactive groups conjugated to the therapeutic oligonucleotides target functionalities present on mobile proteins and covalently bond to the same. Optionally the reactive group is conjugated to the oligonucleotide through the use of a linker group.

Reactive groups of the invention are chemical groups capable of forming a covalent bond with a functionality present on a mobile protein. Reactive groups are coupled or bonded to therapeutic oligonucleotides and corresponding analogs to form modified oligonucleotides. Reactive groups are generally stable in an aqueous environment. The reactive functionalities which are available on mobile proteins for covalent bonding to the chemically reactive group of the modified oligonucleotides and their analogs of the invention are primarily amino groups, carboxyl groups and thiol groups. In one embodiment of the invention, reactive groups include, but are not limited to, reactive double bonds, carboxy, phosphoryl, or convenient acyl groups, either as an ester or a mixed anhydride, or an imidate, thereby capable of forming a covalent bond with functionalities such as amino groups, hydroxy groups or thiol groups at the target site on mobile proteins, in particular on blood proteins. Reactive esters consist of phenolic compounds, thiol esters, alkyl esters, phosphate esters, or the like. In a particularly preferred embodiment of the invention, reactive groups consist of succinimidyl or maleimido groups.

Functionalities of the invention are chemical groups on mobile proteins to which reactive groups on modified oligonucleotides react to form covalent bonds. In another embodiment of the invention, functionalities include but are not limited to, hydroxyl groups for bonding to ester reactive entities; thiol groups for bonding to maleimides and maleimido groups, imidates and thioester groups; amino groups for bonding to carboxy, phosphoryl or acyl groups on reactive entities and carboxyl groups for bonding to amino groups. Such mobile proteins include blood proteins, in particular human serum albumin.

In a particularly preferred embodiment of the invention, the functionality is the free thiol group in human serum albumin.

Linking groups of the invention are chemical moieties that link or conjugate reactive groups to therapeutic oligonucleotides. The linking groups typically contain between four and twelve carbon atoms, saturated or unsaturated and optionally branched. Linking groups include, but are not limited to, one or more alkyl groups such as methyl, ethyl, propyl, butyl, pentyl, hexyl, heptyl, octyl, nonyl, decyl, undecyl, dodecyl, alkoxy groups, alkenyl groups, alkynyl groups or amino group substituted by alkyl groups, cycloalkyl groups, polycyclic groups, aryl groups, polyaryl groups, substituted aryl groups, heterocyclic groups, and substituted heterocyclic groups. Linking groups also comprise polyethoxy amino acids such as AEA ((2-amino) ethoxy acetic acid) or a preferred linking group AEEA ([2-(2-amino)ethoxy)]ethoxy acetic acid).

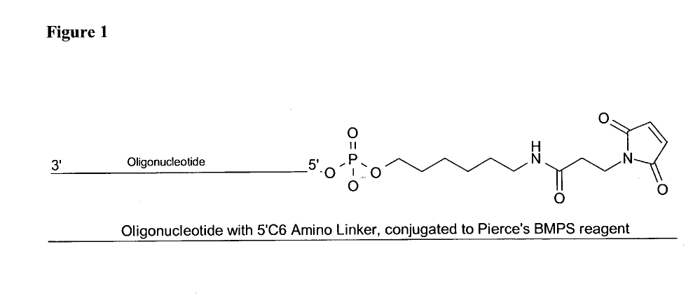

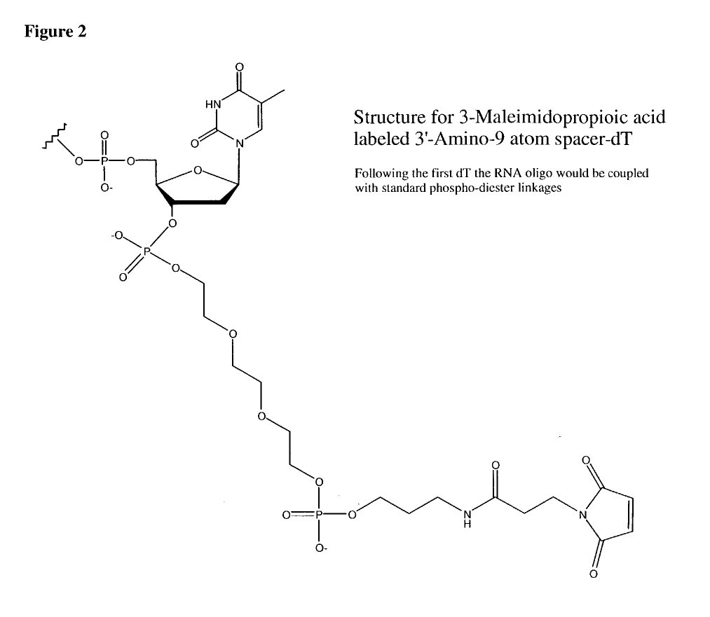

In additionally preferred embodiments, linking groups of the invention consist of the specific linking groups utilized in Examples 1 and 2, infra, for the generation of the MB-003M and MB-006M antisense oligonucleotide and siRNA conjugates. These embodiments are represented in FIGS. 1 and 2 as a 5′ C6 amino linker and a 3′-amino-9 atom spacer (N3_{—}9S-MPA linker), respectively.

Linking groups of the invention may further comprise oligonucleotides which function as linking groups. In one embodiment of the oligonucleotide linking groups of the invention, linking groups comprise between about 4 and about 20 bases that are bonded through phosphodiester or phosphorothioate bonds to either the 5′ or 3′ termini of the therapeutic oligonucleotide. This oligonucleotide linking group may be directly synthesized with a therapeutic oligonucleotide using a DNA synthesizer. Oligonucleotide linking groups of this embodiment may hybridize to the target RNA or DNA in vivo. Alternatively, oligonucleotide linking groups of this embodiment comprise oligonucleotides that do not hybridize to the target RNA or DNA in vivo.

In one embodiment of the oligonucleotide linking groups of the invention, linking groups comprise distinct oligonucleotides containing at least about 15 bases which hybridize to a portion of the therapeutic oligonucleotides of the invention in vitro under stringent hybridization conditions. Oligonucleotide linking groups can comprise at least about 15 bases which hybridize to the target RNA or DNA, in addition to hybridizing to the therapeutic oligonucleotide sequence. Alternatively, oligonucleotide linking groups can comprise oligonucleotides that do not hybridize to the target RNA or DNA under stringent hybridization conditions.

Oligonucleotide linking groups are not restricted by size, but are large enough to allow the therapeutic oligonucleotides to function without interference from the bonded mobile protein. The linking oligonucleotides of the invention are capable of hybridizing to at least a portion of the therapeutic oligonucleotide in vitro under stringent hybridization conditions, and any portion of the linking oligonucleotides may be used to hybridize to the therapeutic oligonucleotide such that the biological activity of the therapeutic oligonucleotide is not inhibited. In addition, a reactive group (e.g., a maleimido or succinimidyl group) is conjugated to another portion of the linking group. Preferably, the reactive group is conjugated to the linking oligonucleotide in a manner that does not interfere with hybridization between the linking oligonucleotide and the therapeutic oligonucleotide.

Hybridization between the oligonucleotide linking group and the therapeutic oligonucleotide specifically occurs in vitro under stringent solution hybridization conditions. The resulting hybrid is available for conjugation in vivo or in vitro to mobile proteins. One of skill in the art understands that stringent hybridization conditions for the generation of in vitro hybrids may vary depending upon numerous factors, such as for example, percent homology between the linking oligonucleotide and the therapeutic oligonucleotide, as well as the length of the respective oligonucleotides. In a non-limiting example, stringent hybridization conditions mean a hybridization which occurs in solution at a temperature of Tm greater than 37° C. One of skill in the art can easily calculate the Tm of oligonucleotide sequences, using equations such as for example the Tm of DNA-DNA hybrids using the following equation: Tm=81.5° C.+16.6(logM)+0.41(% GC)−0.61(% formamide)−500/bp. Similarly, one of skill in the art can easily calculate the Tm of DNA-RNA hybrids using the following equation: Tm=79.8° C.+18.5(logM)+0.58(% GC)+11.8 (% GC)²−0.35(% formamide)−820/L. Likewise, one of skill in the art can easily calculate the Tm of RNA-RNA hybrids using the following equation: Tm=79.8° C.+18.5(logM)+0.58(% GC)+11.8(% GC)²−0.35(% formamide)−820/L. As used in these equations, M is the molarity of monovalent cations, L is the length of the duplex in base pairs and % GC is the percentage of the G and C nucleotides in the DNA. One of skill in the art would understand that these conditions can be modified according to the circumstances of the hybridization.

In a particularly preferred embodiment, the production of therapeutic oligonucleotides of the invention is greatly simplified through the use of oligonucleotide linkers. More specifically, oligonucleotide linking groups having the same sequence and containing reactive groups can be mass-produced. Accordingly, one of skill in the art need merely synthesize or obtain a therapeutic oligonucleotide having at least 15 bases that specifically hybridize to the target RNA or DNA in vivo, and at least 15 bases that hybridize to the mass produced oligonucleotide linking group under stringent hybridization conditions in vitro. Assembly of the modified therapeutic oligonucleotide(s) of the invention consists of hybridizing equivalent concentrations of therapeutic oligonucleotide and oligonucleotide linker in vitro under stringent hybridization conditions, and isolating the resulting hybridization product.

The therapeutic oligonucleotides of the invention, having reactive groups, are capable of forming covalent bonds with mobile proteins. Formation of covalent bonds with mobile proteins has many advantages, such as for example, enhanced half-life and extended efficacy, reduced immune system stimulation, and efficient cell entry. Mobile proteins include, but are not limited to, human serum albumin, human transferrin, human ferritin and human immunoglobulins such as IgM and IgG. In one embodiment of the invention, mobile proteins are targeted which have a half-life circulation of at least about 12 hours. Mobile proteins may be present in a minimum concentration of at least 0.1 μg/ml.

Mobile proteins may consist of the endogenous mobile proteins found in the body. Alternatively, mobile proteins may also consist of recombinantly produced proteins. One of skill in the art is aware of numerous techniques for the in vitro production of recombinant mobile proteins.

In one embodiment of the invention, the therapeutic oligonucleotide-mobile protein conjugate is generated in vivo by the administration of the therapeutic oligonucleotide to the patient, followed by the formation of a covalent bond(s) between the reactive group bonded to the therapeutic oligonucleotide and the functionality present on the mobile protein.

In another embodiment of the invention, the therapeutic oligonucleotide-mobile protein conjugate is generated ex vivo by the formation of a covalent bond(s) between the reactive group bonded to the therapeutic oligonucleotide and the functionality present on the mobile protein. The ex vivo conjugation may also be accomplished by first isolating, purifying or making recombinant forms of an individual mobile protein or limited number of proteins, such as blood proteins, immunoglobulins, human serum albumin, or the like, and combining the protein or proteins ex vivo with the chemically reactive therapeutic oligonucleotide, particularly ASOs. The functionalized blood or mobile protein is then returned to the host to provide, in vivo, the effective therapeutic oligonucleotide conjugates. When conjugates are prepared ex vivo, the ratio of therapeutic oligonucleotides to mobile proteins will vary widely, depending upon factors such as whether whole blood, or a purified component thereof, is used as a bonding site for the therapeutic oligonucleotides.

In one embodiment of the invention, the modified therapeutic oligonucleotides are designed to specifically react with thiol groups on mobile blood proteins. In a particularly preferred embodiment of the invention, such reactions are established through the covalent bonding of a therapeutic oligonucleotide having a reactive maleimide group with a thiol functional group present on a mobile protein, such as for example, human serum albumin or IgG. The maleimide group can be prepared from gamma-maleimido-butyryloxy succinimide ester (GMBS) and maleimidopropionic acid (MPA), using methods known in the art.

The invention provides therapeutic compounds, and a method of specific labeling with maleimide groups. These compounds offer several advantages over non-specific labeling of mobile proteins using groups such as N-hydroxysuccinimide (hereinafter “NHS”) or N-hydroxy-sulfosuccinimide (hereinafter “sulfo-NHS”). Thiol groups are less abundant in vivo than amino groups on endogenous blood proteins. Therefore, fewer maleimide derivatives or compounds labeled with maleimido groups of this invention will covalently bond to proteins. By way of a non-limiting example, human serum albumin (the most abundant blood protein) contains only a single thiol group. Thus, conjugates of therapeutic oligonucleotides derivatized with maleimide, and albumin will tend to comprise approximately a 1:1 molar ratio of the therapeutic oligonucleotide to albumin. In addition to albumin, IgG molecules (class II) also have free thiols. Since IgG molecules and serum albumin constitute the majority of the soluble protein in blood they also make up the majority of the free thiol groups available on blood proteins for covalent bond formation with maleimide-containing therapeutic oligonucleotides.

In addition to providing controlled in vivo labeling, the maleimide-containing therapeutic oligonucleotides of the invention provide specific labeling of human serum albumin and IgG ex vivo. Such ex vivo labeling involves the addition of maleimide-containing therapeutic oligonucleotides to blood, serum or saline solution containing serum albumin and/or IgG. Once modified ex vivo with maleimide-containing therapeutic oligonucleotides, the blood, serum or saline solution can be re-administered to the patient for in vivo treatment.

In a particularly preferred embodiment of the invention, the mobile protein used to generate the therapeutic oligonucleotide conjugate is human serum albumin. The terms “human serum albumin,” “human albumin” and “albumin” as used throughout the application are interchangeable, unless the context indicates otherwise. The amino acid sequence for the preproalbumin form of the human serum albumin protein is:

(SEQ ID NO:23)

MKWVTFISLLFLFSSAYSRGVFRRDAHKSEVAHRFKDLGEENFKALVLIA
FAQYLQQCPFEDHVKLVNEVTEFAKTCVADESAENCDKSLHTLFGDKLCT
VATLRETYGEMADCCAKQEPERNECFLQHKDDNPNLPRLVRPEVDVMCTA
FHDNEETFLKKYLYEIARRHPYFYAPELLFFAKRYKAAFTECCQAADKAA
CLLPKLDELRDEGKASSAKQRLKCASLQKFGERAFKAWAVARLSQRFPKA
EFAEVSKLVTDLTKVHTECCHGDLLECADDRADLAKYICENQDSISSKLK
ECCEKPLLEKSHCIAEVENDEMPADLPSLAADFVESKDVCKNYAEAKDVF
LGMFLYEYARRHPDYSVVLLLRLAKTYETTLEKCCAAADPHECYAKVFDE
FKPLVEEPQNLIKQNCELFEQLGEYKFQNALLVRYTKKVPQVSTPTLVEV
SRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQ
TALVELVKHKPKATKEQLKAVMDDFAAFVEKCCKADDKETCFAEEGKKLV
AASQAALGL

Human serum albumin is synthesized in the liver as a preproalbumin form of the protein. The N-terminal 18 amino acid residues of the preproalbumin form of the protein are subsequently cleaved (amino acids Met1 to Ser18 of SEQ ID NO: 23), and the proalbumin form of the protein is released from the rough endoplasmic reticulum. The proalbumin form of the protein subsequently has the six N-terminal amino acids (amino acids Arg19 to Arg24 of SEQ ID NO: 23) removed in the Golgi vesicles to yield the secreted, mature form of albumin.

Human serum albumin of the invention encompasses the preproalbumin form of the protein, the proalbumin form of the protein, and the mature form of the protein. In a preferred embodiment of the invention, the mature form of human serum albumin is utilized in vivo or ex vivo to generate therapeutic oligonucleotide conjugates.

The invention further contemplates the use of mutants and/or fragments of either the preproalbumin form of the protein (SEQ ID NO: 23), the proalbumin form of the protein, or the mature form of the albumin protein. Fragments of human serum albumin useful with the invention include fragments of 50, 100, 150, 200, 250, 300, 350, 400, 450, 500, or 550 amino acids in length of SEQ ID NO: 23, further including the Cys34 residue. Preferably, fragments of human serum albumin useful with the invention exhibit extended half-lives in vivo, most preferably half-lives comparable to the half-life of endogenous mature human serum albumin.

Mutants of human serum albumin useful in the invention comprise mutants of either the preproalbumin form of the protein (SEQ ID NO: 23), the proalbumin form of the protein, or the mature form of the albumin protein, as well as mutants of any of the fragments recited supra. Mutants of human serum albumin useful in the invention comprise at least one Cysteine residue, preferably Cys34 of the mature form of the albumin protein. Preferably, mutants of human serum albumin contain conservative substitutions such that the overall characteristics, including structure, immunogenicity and half-life, are not substantially changed. One of skill in the art is readily able to determine which amino acids may be substituted to generate mutants. For example, conservative substitutions in mutants of human serum albumin includes substitution of amino acids within the following groups with other amino acids of the same group: replacement of acidic residues such as Asp and Glu; replacement of aromatic residues such as Phe, Tyr, and Trp; replacement of amide residues Asn and Glu; replacement of basic residues Lys, Arg, and His; replacement of hydroxyl residues Ser and Thr; replacement of hydrophobic or aliphatic residues Ala, Val, Leu and Ile; or replacement of small amino acids such as Ala, Ser, Thr, Met and Gly. Strategies for generating mutant proteins are well known to one of skill in the art.

Importantly, amongst free thiol-containing blood proteins, specific labeling with maleimides leads to the preferential formation of ASO-maleimide-albumin conjugates, due to the unique characteristics of albumin itself. The single free thiol group on human serum albumin, highly conserved among species, is the amino acid residue 34 (Cys34) of the mature albumin protein (represented by amino acid 58 of SEQ ID NO: 23).

It has been demonstrated recently that the Cys34 of mature albumin has enhanced reactivity relative to free thiols on other free thiol-containing proteins. This is due in part to the very low pKa value of 5.5 for the Cys34 residue of mature albumin. This value is generally much lower than typical pKa values for cysteine residues of other proteins, which are typically about 8. Due to this low pKa, under normal physiological conditions Cys34 of mature albumin is predominantly in the ionized form, dramatically increasing its reactivity (See U.S. Pat. No. 6,329,336).

Another factor which enhances the reactivity of Cys34 is its location, which is in a crevice close to the surface of one loop of region V of albumin. This location makes Cys34 readily available for reaction with ligands of all kinds, and underscores the biological role of Cys34 as a free radical trap and free thiol scavenger. These properties make Cys34 highly reactive with maleimide moieties, accelerating reaction rates as much as 1000-fold relative to the reaction rates of maleimide with other free-thiol containing proteins.

In contrast to NHS-conjugated therapeutic oligonucleotides, maleimide-containing therapeutic oligonucleotides are generally quite stable in the presence of aqueous solutions and in the presence of free amines. Since maleimide derivatives will only react with free thiols, protective groups are generally not necessary to prevent the maleimide-containing therapeutic oligonucleotides from self-reacting. In addition, the increased stability of the agents permits the use of further purification steps, such as for example HPLC, to prepare highly purified products suitable for in vivo use. Lastly, the increased chemical stability provides a product with a longer shelf life.

By bonding to mobile proteins (in particular blood proteins) with extended serum half-lives, such as immunoglobulin and human serum albumin, a number of advantages ensue. One advantage is that the therapeutic efficacy of the therapeutic oligonucleotides is extended from hours to weeks. Most importantly, conjugation to mobile proteins and blood proteins substantially enhances cellular entry of the therapeutic oligonucleotides, while retaining the binding affinity to the target DNA or RNA.

The complexation with albumin helps reduce immune stimulation by therapeutic oligonucleotides. In addition, complexation with albumin helps avoid non-specific binding of the therapeutic oligonucleotide to endogenous proteins, such as for example plasma proteases. Non-specific binding of therapeutic oligonucleotides to endogenous proteins, particularly plasma proteins, may dispose a patient to diseases such as thrombocytopenia. Non-specific binding of therapeutic oligonucleotides to endogenous proteins also has the more obvious adverse effect of preventing or reducing the amount of therapeutic oligonucleotide reaching its intracellular target. Most importantly, ASO-maleimide-albumin conjugates will be taken up by cells through endocytosis or other pathways related to albumin metabolism. Upon internalization into the cell, with or without albumin degradation in the lysosome, the derivatized ASOs will have access to the target DNA or RNA and achieve the goals of treatment.

In one embodiment of the invention, only one administration of therapeutic oligonucleotide (conjugated or non-conjugated) need be given during the treatment regimen. In another embodiment of the invention, at least two administrations of therapeutic oligonucleotide (conjugated or non-conjugated) may be given during the treatment regimen. One of skill in the art can determine the preferred dosing regimen based upon factors such as the concentration of the therapeutic oligonucleotide, half-life of the conjugated therapeutic oligonucleotide, degree of therapeutic effectiveness for each dose, and other variables.

Uses of the Therapeutic Oligonucleotides of the Invention

The oligonucleotides of the invention may be used to treat diseases in which inhibition of gene expression of a particular gene is beneficial. The diseases include, but are not limited to, cancer, autoimmune diseases, viral and bacterial infections, endocrine system disorders, neural disorders including central and peripheral nervous system disorders, cardiovascular disorders, pulmonary disorders, and reproductive system disorders.

In one particular embodiment of the invention, the therapeutic oligonucleotides of the invention are useful for the amelioration and/or treatment of cancers and other hyperproliferative disorders. Cancer cells are usually characterized by aberrant expression of a gene. Experimental evidence demonstrates that albumin preferentially accumulates in tumors (Clorius, et al., Eur. J. Nucl. Med. 22:989-996 (1995); Wunder, et al. Int. J. Oncol. 11:497-507 (1997)). Furthermore, methotrexate conjugated to albumin exhibited high tumor accumulation and an identical distribution pattern as compared to non-conjugated albumin (Stehle, et al. Anticancer Drugs. 8:677-685, (1997); Stehle, et al. Anticancer Drugs. 8:835-844 (1997)). A phase I trial of albumin conjugated to methotrexate demonstrated an excellent toxicological profile and tumor response in cancer patients, theoretically allowing for outpatient treatment and maintenance of a high quality of life for all cancer patients (Hartung, et al., Clinical Cancer Research, 5:753-759 (1999)). In particular embodiments of the invention, modified therapeutic oligonucleotides comprising at least one of SEQ ID NOs: 1-22 are used to treat human malignancy, and the tumor accumulation demonstrated by the albumin-methotrexate complexes can be exploited for this invention.

Cancers and other hyperproliferative disorders for which this invention provides therapy include, but are not limited to, neoplasms associated with connective and musculoskeletal system tissues, such as fibrosarcoma, rhabdomyosarcoma, myxosarcoma, chondrosarcoma, osteogenic sarcoma, chordoma, and liposarcoma, neoplasms located in the abdomen, bone, brain, breast, colon, digestive system, endocrine glands (adrenal, parathyroid, pituitary, testicles, ovary, thymus, thyroid), eye, head and neck, liver, lymphatic system, nervous system (central and peripheral), pancreas, pelvis, peritoneum, skin, soft tissue, spleen, thorax, and urogenital tract, leukemias (including acute promyelocytic, acute lymphocytic leukemia, acute myelocytic leukemia, myeloblastic, promyelocytic, myelomonocytic, monocytic, erythroleukemia), lymphomas (including Hodgkins and non-Hodgkins lymphomas), multiple myeloma, colon carcinoma, prostate cancer, lung cancer, small cell lung carcinoma, bronchogenic carcinoma, testicular cancer, cervical cancer, ovarian cancer, breast cancer, angiosarcoma, lymphangiosarcoma, endotheliosarcoma, lymphangioendotheliosarcoma, synovioma, mesothelioma, Ewing's sarcoma, leiomyosarcoma, squamous cell carcinoma, basal cell carcinoma, pancreatic cancer, renal cell carcinoma, Wilm's tumor, hepatoma, bile duct carcinoma, adenocarcinoma, epithelial carcinoma, melanoma, sweat gland carcinoma, sebaceous gland carcinoma, papillary carcinoma, papillary adenocarcinoma, glioma, astrocytoma, medulloblastoma, craniopharyngioma, ependymoma, pinealoma, emangioblastoma, acoustic neuroma, oligodendroglioma, menangioma, neuroblastoma, retinoblastoma, bladder carcinoma, embryonal carcinoma, cystadenocarcinoma, medullary carcinoma, choriocarcinoma, and seminoma.

A method of treatment according to one embodiment of the invention comprises the steps of preparing modified therapeutic oligonucleotides with a maleimide group and optionally a linker; administering a therapeutically effective amount of the modified therapeutic oligonucleotides in human patients to form albumin conjugates; cellular entry of the albumin conjugates through endocytosis or other mechanisms, and; specific hybridization of the therapeutic oligonucleotide with the aberrantly expressed gene, thereby inhibiting gene expression.

In one embodiment of the invention, administration is conducted immediately following the detection of the presence of cancerous cells, in order to maintain the oligonucleotides in the blood stream. The therapeutic oligonucleotides can also be used in combination with surgery to catch the window of migration and prevent further metastatic action via the blood. The oligonucleotides of the invention also could be used at chronic or late stage cancers to reduce further metastatic spread of any cancer.

In an alternative embodiment of the invention, the therapeutic oligonucleotides of the invention are used in a prophylactic manner to prevent diseases or disorders in individuals demonstrating a pre-disposition to development of the same diseases or disorders.

The modified therapeutic oligonucleotides are administered in a physiologically acceptable medium (e.g., deionized water, phosphate buffered saline (PBS), saline, aqueous ethanol or other alcohol, plasma, proteinaceous solutions, mannitol, aqueous glucose, vegetable oil, or the like). Buffers may also be included, particularly where the media are generally buffered at a pH in the range of about 5 to 10, where the buffer will generally range in concentration from about 50 to 250 mM, salt, where the concentration of salt will generally range from about 5 to 500 mM, physiologically acceptable stabilizers, and the like. The compounds may be lyophilized for convenient storage and transport.

The blood of the mammalian host may be monitored for the activity of the therapeutic oligonucleotides and/or presence of the modified therapeutic oligonucleotides. By taking a portion or sample of the blood of the host at different times, one may determine whether the therapeutic oligonucleotide has become bound to the long-lived mobile proteins in sufficient concentration to be therapeutically effective and, thereafter, the level of therapeutic oligonucleotide in the blood. If desired, one may also identify the particular mobile proteins to which the therapeutic oligonucleotide is bound. This is particularly important when using non-specific therapeutic oligonucleotides. For specific maleimide-containing therapeutic oligonucleotides, it is much simpler to calculate the half-life of serum albumin and IgG.

### EXAMPLES

### Example 1

Antisense Oligonucleotide Synthesis

Specific antisense oligonucleotides, MB003 and MB006, were synthesized with a phosphorothioate backbone by Trilink Biotechnologies (San Diego, Calif.). ASOs MB003 and MB006 were directed to mRNA transcripts of the human bcl-xl gene (See Simoes-Wust et al., Int. J. Cancer, 87:582-90 (2000); U.S. Pat. No. 6,214,986). Overexpression of the human bcl-xl gene has been associated with a spectrum of cancer cells. Accordingly, treatment with ASOs specific for the human bcl-xl gene should reduce or eliminate the proliferation or growth of various cancerous cells. The sequences of the MB003 and MB006 ASOs is set forth below:

| | |
|---|---|
| MB-003: | |
| 5′-AAAGTATCCCAGCCGCCGTT-3′ | (SEQ ID NO:21) |
| MB-006: | |
| 5′-TCCCGGTTGCTCTGAGACAT-3′ | (SEQ ID NO:22) |

In addition, two ASOs were synthesized which contained a 5′-(BMPS)(C6NH) linker and maleimide reactive moiety, as shown in FIG. 1. These two ASOs, identified as MB-003M and MB-006M, have a 5′ six-carbon linker conjugated to Pierce's N-(β-maleidoproploxy) succinimide ester (BMPS) reactive reagent. Accordingly, the structures of MB-003M and MB-006M are represented by the following:

| | |
|---|---|
| MB-003M: | (SEQ ID NO:21) |

5′-(BMPS)(C6NH)AAAGTATCCCAGCCGCCGTT-3′

| | |
|---|---|
| MB-006M: | (SEQ ID NO:22) |

5′-(BMPS)(C6NH)TCCCGGTTGCTCTGAGACAT-3′

Phosphorothioate oligonucleotides were synthesized using standard phosphoramidite chemistry (Gait, M. J., “Oligonucleotide Synthesis, a practical approach”, Oxford Univ. Press, New York (1984)) on an ABI Expedite 8909 DNA synthesizer. After deprotection with AMA (Reddy, M. P. et al., Tetrahedron Lett., 35(25):4311-14 (1994)), the oligos were purified by reverse phase HPLC using a gradient of acetonitrile in 50 mM triethylammonium acetate on a Waters μBondapak C-18 cartridge.

The linker modified oligonucleotides were synthesized in a similar fashion, with the exception that a C-6 amino linker was added to the 5′ end of the oligonucleotides using MMT-C-6 amino linker phosphoramidite (Trilink Biotechnologies, San Diego, Calif.). After purification and removal of the protecting group from the amine, the oligonucleotide was conjugated to N-(β-maleimidopropyloxy) succinimide ester (BMPS) (Pierce, Milwaukee, Wis.) using manufacturer recommended conditions. The excess BMPS was removed using a 10 ml bed size exclusion column (LH-20, Pharmacia, Piscataway, N.J.).

HSA-Conjugate preparation

Following synthesis of the MB-003M and MB-006M modified ASOs, protein conjugates are formed between human serum albumin (HSA) and the MB-003M and MB-006M ASOs. More specifically, MB-003M and MB-006M are each added to react with HSA, separately and respectively, and incubated at 37° C. to facilitate formation of the conjugates. Synthesis of HSA-conjugates is verified by SDS-PAGE and HPLC to determine the degree of conjugation. Successful formation of conjugates between the MB-003M and MB-006M ASOs, and HSA is evidenced by a change in the gel or HPLC profiles compared to un-reacted HSA.

In vitro Stability Assessment

The in vitro stability of the MB-003M and MB-006M HSA conjugates against nuclease degradation is tested. Specifically, HSA-conjugates are incubated with endonuclease(s) for a specified period of time and at a specified set of reaction parameters. Following incubation, samples are analyzed using SDS-PAGE and HPLC, and compared to samples that are not challenged with nuclease activity.

Assessment of in vitro Activity

The specific activity of the MB-003M and MB-006M HSA conjugates is tested in vitro by incubating selected cancer cells lines with the MB-003M and MB-006M HSA conjugates, respectively, and determining the effects of the conjugate activity as compared to untreated control cells. Reduction and/or inhibition of overexpression of the human bcl-xl gene indicates that the MB-003M and/or MB-006M HSA conjugates have effective ASO activity in conjugated form.

### Example 2

siRNA Conjugates of the Invention

A therapeutic oligonucleotide of the invention consisting of a modified siRNA having a reactive group is synthesized. Initially, the “sense” strand of the duplex RNA is synthesized using known techniques. The “sense” strand is synthesized with the addition of a N3_{—}9S-MPA linker (FIG. 2) at the 3′ terminus. The synthesized strand having the linker is recovered and purified. Another RNA strand complementary to the sense strand is synthesized using known techniques, recovered and purified.

After purification, the “sense” RNA strand and the complementary RNA strand are annealed to generate an siRNA duplex molecule. Following annealing, any additional purification steps deemed necessary are carried out.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is herein incorporated by reference. Further, the hard copy of the sequence listing submitted herewith and the corresponding computer readable form (CRF) are both incorporated herein by reference in their entireties.

Certain therapeutic oligonucleotides of the present invention, as well as methods of treatment using the same therapeutic oligonucleotides, were disclosed in U.S. Provisional Patent Application Serial No. 60/356,053, filed Feb. 13, 2002, which is herein incorporated by reference in its entirety.

What is claimed is:

1. A method of treating a disease by administering a therapeutic oligonucleotide to a patient in need thereof, comprising administering to a patient a therapeutic oligonucleotide including a reactive group, the reactive group upon reaction with a mobile protein forming a covalent bond through which said mobile protein is conjugated to said therapeutic oligonucleotide.

2. The method of claim 1, wherein the said reactive group is bonded to the oligonucleotide through a linking group.

3. The method of claim 2, wherein the said linking group is selected from the group consisting of:
(a) an alkyl group;
(b) an alkoxy group;
(c) an alkenyl group;
(d) an alkynyl group;
(e) an amino group substituted by an alkyl group;
(f) a cycloalkyl group;
(g) a polycyclic group;
(h) a substituted heterocyclic group;
(i) polyethoxy amino acids;
(j) a peptide nucleic acid;
(k) a 5′ C6 amino linker; and
(l) a 3′ amino-9 atom linker.

4. The method of claim 3, wherein the linking group comprises between 4 and 12 carbon atoms.

5. The method of claim 3, wherein the linking group comprises polyethoxy amino acids.

6. The method of claims 2 or 3, wherein the said linking group comprises an oligonucleotide that hybridizes to at least 15 bases of a portion of a DNA or RNA target.

7. The method of claims 2 or 3, wherein the said linking group comprises an oligonucleotide that, when placed in stringent hybridization conditions, hybridizes to at least 15 bases of a portion of the therapeutic oligonucleotide.

8. The method of claims 2 or 3, wherein the said linking group comprises an oligonucleotide that does not hybridize to a DNA or RNA target.

9. The method of claims 1 or 2, wherein the reactive group is selected from the group consisting of:
(a) a succinimidyl group;
(b) a maleimido group;
(c) a hydrazine group; and
(d) a carbonyl group.

10. The method of claim 9, wherein the reactive group is a maleimido group.

11. The method of claim 1, wherein the mobile protein is a blood protein.

12. The method of claim 11, wherein the blood protein is selected from the group consisting of:
(a) human serum albumin protein;
(b) human transferrin protein;
(c) human ferritin protein; and
(d) human immunoglobulin proteins.

13. The method of claim 12, wherein the blood protein is human serum albumin protein.

14. The method of claim 1, wherein the disease is a hyperproliferative disorder.

15. The method of claim 1, wherein the disease is an autoimmune disorder.

16. The method of claim 1, wherein the disease is a viral infection.

17. The method of claim 1, wherein the disease is a bacterial infection.

18. The method of claim 1, wherein the disease is an endocrine disorder.

19. The method of claim 1, wherein the disease is a neural disorder.

20. The method of claim 1, wherein the disease is a cardiovascular disorder.

21. The method of claim 1, wherein the disease is a pulmonary disorder.

22. The method of claim 1, wherein the disease is a reproductive system disorder.

23. The method of claim 1, wherein the reactive group is capable of forming a covalent bond with a mobile protein in vivo.

24. The method of claim 1, wherein the reactive group is capable of forming a covalent bond with a mobile protein ex vivo.

25. The method of claim 1, comprising administering a composition of matter comprising said therapeutic oligonucleotide and a pharmaceutically acceptable carrier.

26. The method of claims 12 or 13, wherein the human serum albumin protein is a naturally occurring human serum albumin protein.

27. The method of claims 12 or 13, wherein the human serum albumin protein is a recombinant human serum albumin protein.

28. The method of claims 12 or 13, wherein the human serum albumin protein is a fragment of SEQ ID NO: 23.

29. The method of claims 12 or 13, wherein the human serum albumin protein is a variant of SEQ ID NO: 23.

30. A method of treating a disease by administering a therapeutic oligonucleotide to a patient in need thereof, comprising administering to a patient a therapeutic oligonucleotide conjugated by a covalent bond with a mobile protein.

31. The method of claim 30, wherein the said reactive group is bonded to the oligonucleotide through a linking group.

32. The method of claim 31, wherein the said linking group is selected from the group consisting of:
(a) an alkyl group;
(b) an alkoxy group;
(c) an alkenyl group;
(d) an alkynyl group;
(e) an amino group substituted by an alkyl group;
(f) a cycloalkyl group;
(g) a polycyclic group;
(h) a substituted heterocyclic group;
(i) polyethoxy amino acids;
(j) a peptide nucleic acid;
(k) a 5′ C6 amino linker; and
(l) a 3′ amino-9 atom linker.

33. The method of claim 32, wherein the linking group comprises between 4 and 12 carbon atoms.

34. The method of claim 32, wherein the linking group comprises polyethoxy amino acids.

35. The method of claims 31 or 32, wherein the said linking group comprises an oligonucleotide that hybridizes to at least 16 bases of a portion of a DNA or RNA target.

36. The method of claims 31 or 32, wherein the said linking group comprises an oligonucleotide that, when placed in stringent hybridization conditions, hybridizes to at least 16 bases of a portion of the therapeutic oligonucleotide.

37. The method of claims 31 or 32, wherein the said linking group comprises an oligonucleotide that does not hybridize to a DNA or RNA target.

38. The method of claims 30 or 31, wherein the reactive group is selected from the group consisting of:
(a) a succinimidyl group;
(b) a maleimido group;
(c) a hydrazine group; and
(d) a carbonyl group.

39. The method of claim 38, wherein the reactive group is a maleimido group.

40. The method of claim 30, wherein the mobile protein is a blood protein.

41. The method of claim 40, wherein the blood protein is selected from the group consisting of:
(a) human serum albumin protein;
(b) human transferrin protein;
(c) human ferritin protein; and
(d) human immunoglobulin proteins.

42. The method of claim 41, wherein the blood protein is human serum albumin protein.

43. The method of claim 30, wherein the disease is a hyperproliferative disorder.

44. The method of claim 30, wherein the disease is an autoimmune disorder.

45. The method of claim 30, wherein the disease is a viral infection.

46. The method of claim 30, wherein the disease is a bacterial infection.

47. The method of claim 30, wherein the disease is an endocrine disorder.

48. The method of claim 30, wherein the disease is a neural disorder.

49. The method of claim 30, wherein the disease is a cardiovascular disorder.

50. The method of claim 30, wherein the disease is a pulmonary disorder.

51. The method of claim 30, wherein the disease is a reproductive system disorder.

52. The method of claim 30, wherein the reactive group is capable of forming a covalent bond with a mobile protein in vivo.

53. The method of claim 30, wherein the reactive group is capable of forming a covalent bond with a mobile protein ex vivo.

54. The method of claim 30, comprising administering a composition of matter comprising said therapeutic oligonucleotide and a pharmaceutically acceptable carrier.

55. The method of claims 31 or 32, wherein the human serum albumin protein is a naturally occurring human serum albumin protein.

56. The method of claims 31 or 32, wherein the human serum albumin protein is a recombinant human serum albumin protein.

57. The method of claims 41 or 42, wherein the human serum albumin protein is a fragment of SEQ ID NO: 23.

58. The method of claims 41 or 42, wherein the human serum albumin protein is a variant of SEQ ID NO: 23.

59. A method of treating a disease, comprising administering to a patient in need thereof a double-stranded RNA duplex including a reactive group, the reactive group upon reaction with a mobile protein forming a covalent bond through which said mobile protein is conjugated to said double-stranded RNA duplex.

60. The method of claim 59, wherein each of the strands of the double-stranded RNA duplex is 15-30 bases in length.

61. The method of claim 59, wherein the sequence of one strand of the RNA duplex shares at least 90% homology with 15-30 bases of a portion of a RNA or DNA target.

62. The method of claim 59, wherein the sequence of one strand of the RNA duplex shares 100% homology with 15-30 bases of a portion of a RNA or DNA target.

63. The method of claim 59, wherein the RNA duplex directs nuclease cleavage of the target RNA.

64. The method of claim 59, wherein at least one of the 3′ termini of the RNA duplex contains an overhanging sequence of between one and three bases.

65. The method of claim 59, wherein both 3′ termini of the RNA duplex contain overhanging sequence of between one and three bases.

66. The method of claims 64 or 65, wherein the 3′ termini of the RNA duplex consists of two overhanging bases.

67. The method of claim 59, wherein the said reactive group is bonded to the double stranded RNA duplex at any one of the four duplex termini.

68. The method of claim 59, wherein the said reactive group is bonded to the oligonucleotide through a linking group.

69. The method of claim 68, wherein the said linking group is selected from the group consisting of:
(a) an alkyl group;
(b) an alkoxy group;
(c) an alkenyl group;
(d) an alkynyl group;
(e) an amino group substituted by an alkyl group;
(f) a cycloalkyl group;
(g) a polycyclic group;
(h) a substituted heterocyclic group;
(i) polyethoxy amino acids;
(j) a peptide nucleic acid;
(k) a 5′ C6 amino linker; and
(l) a 3′ amino-9 atom linker.

70. The method of claim 69, wherein the linking group comprises between 4 and 12 carbon atoms.

71. The method of claim 69, wherein the linking group comprises polyethoxy amino acids.

72. The method of claims 59 or 68, wherein the reactive group is selected from the group consisting of:
(a) a succinimidyl group;
(b) a maleimido group;
(c) a hydrazine group; and
(d) a carbonyl group.

73. The method of claim 72, wherein the reactive group is a maleimido group.

74. The method of claim 59, wherein the mobile protein is a blood protein.

75. The method of claim 74, wherein the blood protein is selected from the group consisting of:
(a) human serum albumin protein;
(b) human transferrin protein;
(c) human ferritin protein; and
(d) human immunoglobulin proteins.

76. The method of claim 75, wherein the blood protein is human serum albumin protein.

77. The method of claim 59, wherein the disease is a hyperproliferative disorder.

78. The method of claim 59, wherein the disease is an autoimmune disorder.

79. The method of claim 59, wherein the disease is a viral infection.

80. The method of claim 59, wherein the disease is a bacterial infection.

81. The method of claim 59, wherein the disease is an endocrine disorder.

82. The method of claim 59, wherein the disease is a neural disorder.

83. The method of claim 59, wherein the disease is a cardiovascular disorder.

84. The method of claim 59, wherein the disease is a pulmonary disorder.

85. The method of claim 59, wherein the disease is a reproductive system disorder.

86. The method of claim 59, wherein the reactive group is capable of forming a covalent bond with a mobile protein in vivo.

87. The method of claim 59, wherein the reactive group is capable of forming a covalent bond with a mobile protein ex vivo.

88. The method of claim 59, comprising administering a composition of matter comprising said double-stranded RNA duplex and a pharmaceutically acceptable carrier.

89. The method of claims 75 or 76, wherein the human serum albumin protein is a naturally occurring human serum albumin protein.

90. The method of claims 75 or 76, wherein the human serum albumin protein is a recombinant human serum albumin protein.

91. The method of claims 75 or 76, wherein the human serum albumin protein is a fragment of SEQ ID NO: 23.

92. The method of claims 75 or 76, wherein the human serum albumin protein is a variant of SEQ ID NO: 23.

93. A method of treating a disease, comprising administering to a patient in need thereof a double-stranded RNA duplex conjugated by a covalent bond with a mobile protein.

94. The method of claim 93, wherein each of the strands of the double-stranded RNA duplex is 15-30 bases in length.

95. The method of claim 93, wherein the sequence of one strand of the RNA duplex shares at least 90% homology with 15-30 bases of a portion of a RNA or DNA target.

96. The method of claim 93, wherein the sequence of one strand of the RNA duplex shares 100% homology with 15-30 bases of a portion of a RNA or DNA target.

97. The method of claim 93, wherein the RNA duplex directs nuclease cleavage of the target RNA.

98. The method of claim 93, wherein at least one of the 3′ termini of the RNA duplex contains an overhanging sequence of between one and three bases.

99. The method of claim 93, wherein both 3′ termini of the RNA duplex contain overhanging sequence of between one and three bases.

100. The method of claims 98 or 99, wherein the 3′ termini of the RNA duplex consists of two overhanging bases.

101. The method of claim 93, wherein the said reactive group is bonded to the double stranded RNA duplex at any one of the four duplex termini.

102. The method of claim 93, wherein the said reactive group is bonded to the oligonucleotide through a linking group.

103. The method of claim 102, wherein the said linking group is selected from the group consisting of:
(a) an alkyl group;
(b) an alkoxy group;
(c) an alkenyl group;
(d) an alkynyl group;
(e) an amino group substituted by an alkyl group;
(f) a cycloalkyl group;
(g) a polycyclic group;
(h) a substituted heterocyclic group;
(i) polyethoxy amino acids;
(j) a peptide nucleic acid;
(k) a 5′ C6 amino linker; and
(l) a 3′ amino-9 atom linker.

104. The method of claim 103, wherein the linking group comprises between 4 and 12 carbon atoms.

105. The method of claim 103, wherein the linking group comprises polyethoxy amino acids.

106. The method of claims 93 or 102, wherein the reactive group is selected from the group consisting of:
(a) a succinimidyl group;
(b) a maleimido group;
(c) a hydrazine group; and
(d) a carbonyl group.

107. The method of claim 106, wherein the reactive group is a maleimido group.

108. The method of claim 93, wherein the mobile protein is a blood protein.

109. The method of claim 108, wherein the blood protein is selected from the group consisting of:
(a) human serum albumin protein;
(b) human transferrin protein;
(c) human ferritin protein; and
(d) human immunoglobulin proteins.

110. The method of claim 109, wherein the blood protein is human serum albumin protein.

111. The method of claim 93, wherein the disease is a hyperproliferative disorder.

112. The method of claim 93, wherein the disease is an autoimmune disorder.

113. The method of claim 93, wherein the disease is a viral infection.

114. The method of claim 93, wherein the disease is a bacterial infection.

115. The method of claim 93, wherein the disease is an endocrine disorder.

116. The method of claim 93, wherein the disease is a neural disorder.

117. The method of claim 93, wherein the disease is a cardiovascular disorder.

118. The method of claim 93, wherein the disease is a pulmonary disorder.

119. The method of claim 93, wherein the disease is a reproductive system disorder.

120. The method of claim 93, wherein the reactive group is capable of forming a covalent bond with a mobile protein in vivo.

121. The method of claim 93, wherein the reactive group is capable of forming a covalent bond with a mobile protein ex vivo.

122. The method of claim 93, comprising administering a composition of matter comprising said double-stranded RNA duplex and a pharmaceutically acceptable carrier.

123. The method of claims 109 or 110, wherein the human serum albumin protein is a naturally occurring human serum albumin protein.

124. The method of claims 109 or 110, wherein the human serum albumin protein is a recombinant human serum albumin protein.

125. The method of claims 109 or 110, wherein the human serum albumin protein is a fragment of SEQ ID NO: 23.

126. The method of claims 109 or 110, wherein the human serum albumin protein is a variant of SEQ ID NO: 23.

127. A therapeutic oligonucleotide of 15-30 bases in length comprising a portion that binds an RNA or DNA target, further including a reactive group bonded to the oligonucleotide, the reactive group upon reaction with a mobile protein forming a covalent bond through which said mobile protein is conjugated to said therapeutic oligonucleotide.

128. A therapeutic oligonucleotide comprising 15-30 bases in length including a reactive group, the reactive group upon reaction with a mobile protein forming a covalent bond with said mobile protein, wherein the oligonucleotide is selected from the group consisting of SEQ ID NOs: 1-22.

129. A therapeutic oligonucleotide of 15-30 bases in length comprising a portion that binds a RNA or DNA target, wherein said oligonucleotide is conjugated by a covalent bond with a mobile protein.

130. A therapeutic oligonucleotide comprising a double stranded RNA duplex including a reactive group, the reactive group upon reaction with a mobile protein forming a covalent bond through which said mobile protein is conjugated to said double-stranded RNA duplex.

131. A therapeutic oligonucleotide comprising a double stranded RNA duplex conjugated by a covalent bond with a mobile protein.


SEQUENCE LISTING (23 sequences)

SEQ ID NO: 1 - DNA, 20 bp, Artificial Sequence
  Antisense oligonucleotide directed against murine ICAM-1
tgcatcccccaggccaccat

SEQ ID NO: 2 - DNA, 20 bp, Artificial Sequence
  Antisense oligonucleotide directed against human ICAM-1
gcccaagctggcatccgtca

SEQ ID NO: 3 - DNA, 15 bp, Artificial Sequence
  Antisense oligonucleotide directed against human erb-B-2
ggtgctcactgcggc

SEQ ID NO: 4 - DNA, 16 bp, Artificial Sequence
  Antisense oligonucleotide directed against human c-myc
aaccgttgaggggcat

SEQ ID NO: 5 - DNA, 15 bp, Artificial Sequence
  Antisense oligonucleotide directed against human c-myc
aacgttgaggggcat

SEQ ID NO: 6 - DNA, 16 bp, Artificial Sequence
  Antisense oligonucleotide directed against human c-myc
taacgttgaggggcat

SEQ ID NO: 7 - DNA, 24 bp, Artificial Sequence
  Antisense oligonucleotide directed against human c-myb
tatgctgtgccggggtcttcgggc

SEQ ID NO: 8 - DNA, 18 bp, Artificial Sequence
  Antisense oligonucleotide directed against human c-myb
gtgccggggtcttcgggc

SEQ ID NO: 9 - DNA, 18 bp, Artificial Sequence
  Antisense oligonucleotide directed against human IGF-1R
ggaccctcctccggagcc

SEQ ID NO: 10 - DNA, 18 bp, Artificial Sequence
  Antisense oligonucleotide directed against human IGF-1R
tcctccggagccagactt

SEQ ID NO: 11 - DNA, 15 bp, Artificial Sequence
  Antisense oligonucleotide directed against human EGFR
ccgtggtcatgctcc

SEQ ID NO: 12 - DNA, 21 bp, Artificial Sequence
  Antisense oligonucleotide directed against human VEGF
cagcctggctcaccgccttgg

SEQ ID NO: 13 - DNA, 20 bp, Artificial Sequence
  Antisense oligonucleotide directed against murine PKC-alpha
cagccatggttccccccaac

SEQ ID NO: 14 - DNA, 20 bp, Artificial Sequence
  Antisense oligonucleotide directed against human PKC-alpha
gttctcgctggtgagtttca

SEQ ID NO: 15 - DNA, 18 bp, Artificial Sequence
  Antisense oligonucleotide directed against human bcl-2
tctcccagcgtgcgccat

SEQ ID NO: 16 - DNA, 15 bp, Artificial Sequence
  Antisense oligonucleotide directed against human c-raf-1 protein kinase
gtgctccattgatgc

SEQ ID NO: 17 - RNA, 33 nt, Artificial Sequence
  Ribozyme directed against human VEGF-1
gaguucugaugaggccgaaaggccgaaagucug

SEQ ID NO: 18 - DNA, 20 bp, Artificial Sequence
  Antisense oligonucleotide directed against human P53
ccctgctcccccctggctcc

SEQ ID NO: 19 - DNA, 18 bp, Artificial Sequence
  Antisense oligonucleotide directed against human urokinase recept or
cggcgggtgacccatgtc

SEQ ID NO: 20 - DNA, 25 bp, Artificial Sequence
  Antisense oligonucleotide directed against human HIV-1
tcttcctctctctacccacgctctc

SEQ ID NO: 21 - DNA, 20 bp, Artificial Sequence
  MB-003 antisense oligonucleotide
aaagtatcccagccgccgtt

SEQ ID NO: 22 - DNA, 20 bp, Artificial Sequence
  MB006 antisense oligonucleotide
tcccggttgctctgagacat

SEQ ID NO: 23 - protein, 609 aa, Homo sapiens [sequence omitted]